(19) United States
(12) Patent Application Publication
(10) Pub. No.: US 2012/0065689 A1
(43) Pub. Date: Mar. 15, 2012

(54) Joint Fusion Construct And Method

(76) Inventors: Priya Prasad, Miami, FL (US); Mark S. Myerson, Baltimore, MD (US); Sanjoy Choudhuri, Miami, FL (US)

(21) Appl. No.: 12/879,224

(22) Filed: Sep. 10, 2010

(51) Int. Cl. A61B 17/80

(52) U.S. Cl. 606/286

(57) ABSTRACT

A construct for fusing a joint between a proximal bone and a distal bone comprises a plate body having a proximal end, a distal end, and a longitudinal axis extending between the proximal and distal ends; a rigid joint bridge on the plate body intermediate the proximal and distal ends, the bridge separating the plate body between a proximal bone portion and a distal bone portion; a plurality of screw receiving holes extending through the proximal bone portion of the plate, the distal most pair of the holes defining a first transverse axis at an oblique angle relative the longitudinal axis; a plurality of screw receiving holes extending through the distal bone portion of the plate, the proximal most pair of the holes defining a second transverse axis at an oblique angle relative the longitudinal axis; a plurality of screws extending through the holes and attaching the plate body to the proximal and distal bones; and a joint screw traversing the joint between the first and second transverse axes.

### BACKGROUND

The present invention relates in general to orthopedic devices and procedures, and more particularly to a bone plate and screw construct for fusing joints.

In some cases patients require surgery to fuse their bone joints. For instance, the MTP (metatarsal-phalangeal) joint of the big toe is the main joint at the base of the toe. This joint might become painful and stiff as a result of arthritis, a longstanding bunion deformity, previous injury, or other ailment. Surgical options are often sought when pain in the joint becomes significant enough to interfere with activities of daily life. A bony ridge around the joint might also become prominent enough to interfere with the wearing of certain shoes.

MTP fusion is a surgical procedure where the joint is permanently fixed, creating a fusion between the two bones adjacent to the joint. The goals of the surgery are to reduce pain and discomfort and to improve shoe fit by eliminating the last few degrees of painful movement and removing the bony ridges adjacent to the joint. Mobility of the joint is, however, permanently sacrificed in the process. Arthritis or damage to the joint generally is not in itself a reason for surgery, but it might be needed if pain becomes so bad that a completely stiff joint with less pain would be preferable.

A MTP joint fusion surgery can be performed under general, spinal or epidural anesthetic, or with only the involved foot anaesthetized. A cut is made over the joint, so the bony ridges next to the joint and the remaining joint cartilage can be removed. The big toe is positioned in the most comfortable position next to the second toe and stabilized, usually with small metal plates or screws, to hold the bones in place until fused together. The procedure normally takes less than an hour in uncomplicated cases.

While joint fusion surgeries are known, no one has previously made or used a plate and screw construct for fusing joints in accordance with this disclosure.

### BRIEF DESCRIPTION OF DRAWINGS

While the specification concludes with claims which particularly point out and distinctly claim the invention, it is believed the invention will be better understood from the following description taken in conjunction with the accompanying drawings illustrating some non-limiting examples of the invention. Unless otherwise indicated, the figures are not necessarily drawn to scale, but rather to illustrate the principles of the invention.

FIG. 1 depicts plan views of an example of left and right joint fusion plates;

FIG. 2 depicts a lateral view of a joint fusion plate positioned on a metatarsal and phalanx;

FIG. 3 depicts a perspective view of a joint fusion plate secured to a metatarsal and phalanx with screws; and

FIG. 4 depicts a perspective view of a joint fusion plate secured to a metatarsal and phalanx with screws with a screw traversing the joint.

### SUMMARY

A construct for fusing a joint between a proximal bone and a distal bone may comprise a plate body having a proximal end, a distal end, and a longitudinal axis extending between the proximal and distal ends. A rigid joint bridge may be positioned intermediate the proximal and distal ends with the bridge separating the plate body between a proximal bone portion and a distal bone portion. A plurality of screw receiving holes may extend through the proximal bone portion of the plate with the distal most pair of the holes defining a first transverse axis at an oblique angle relative the longitudinal axis. A plurality of screw receiving holes may extend through the distal bone portion of the plate with the proximal most pair of the holes defining a second transverse axis at an oblique angle relative the longitudinal axis. A plurality of screws may extend through the holes and attach the plate body to the proximal and distal bones. A joint screw may traverse the joint between the first and second transverse axes.

The construct may be used where the proximal bone is the first metatarsal and the distal bone is the first phalanx. The proximal and distal bone portions may be angled relative one another. The distal bone may be fused approximately at or between about 8 and about 20 degrees of dorsiflexion with respect to the first metatarsal. The distal bone may be fused approximately at or between about 1 and about 9 degrees of valgus with respect to the 1^{st} metatarsal.

A fusion plate for a joint between a proximal bone and a distal bone may comprise a plate body having a proximal end, a distal end, and a longitudinal axis extending between the proximal and distal ends. A rigid joint bridge on the plate body may be positioned intermediate the proximal and distal ends with the bridge separating the plate body between a proximal bone portion and a distal bone portion. A plurality of screw receiving holes may extend through the proximal bone portion of the plate with the distal most pair of the holes defining a first transverse axis at an oblique angle relative the longitudinal axis. A plurality of screw receiving holes may extend through the distal bone portion of the plate with the proximal most pair of the holes defining a second transverse axis at an oblique angle relative the longitudinal axis. The first and second transverse axes may define a channel to receive a screw traversing the joint.

The distal most and proximal most pairs of holes may be threaded fixed angle screw holes. The proximal bone portion may comprise a slotted screw hole. The proximal bone portion may comprise a bendable proximal tab comprising a screw receiving hole. The distal bone portion may comprise a bendable distal tab comprising a screw receiving hole. In one embodiment the screw receiving holes consist of three threaded screw holes and one slotted screw hole in the proximal bone portion, and three threaded screw holes in the distal bone portion.

A method for fusing the metatarsal-phalangeal joint may comprise traversing the metatarsal-phalangeal joint with a joint screw; obtaining the fusion plate; placing the fusion plate on the metatarsal and the phalanx such that the bridge spans the metatarsal-phalangeal joint, and the joint screw is positioned within the channel; obtaining a plurality of screws sized for the screw receiving holes; attaching the proximal bone portion to the metatarsal with one or more screws; attaching the distal bone portion to the phalange with one or more screws; and obtaining a joint screw. Optionally, the step of traversing is performed after the steps of attaching the plate.

A fusion plate for the metatarsal-phalangeal joint may comprise a plate body having a proximal end, a distal end, and a longitudinal axis extending between the proximal and distal ends. A rigid joint bridge on the plate body may be intermediate the proximal and distal ends with the bridge separating the plate body between a metatarsal portion and a phalanx portion. The metatarsal and phalanx portions may be anglee relative one another in the dorsal direction. Three or more screw receiving holes may extend through the metatarsal portion of the plate, with the distal most pair of the holes defining a first transverse axis at an oblique angle between about 50 and about 60 degrees relative the longitudinal axis. Three of more of screw receiving holes may extend through the phalanx portion of the plate with the proximal most pair of the holes defining a second transverse axis substantially parallel with the first transverse axis. The first and second transverse axes may define a channel adapted to receive a screw traversing the joint.

### DETAILED DESCRIPTION

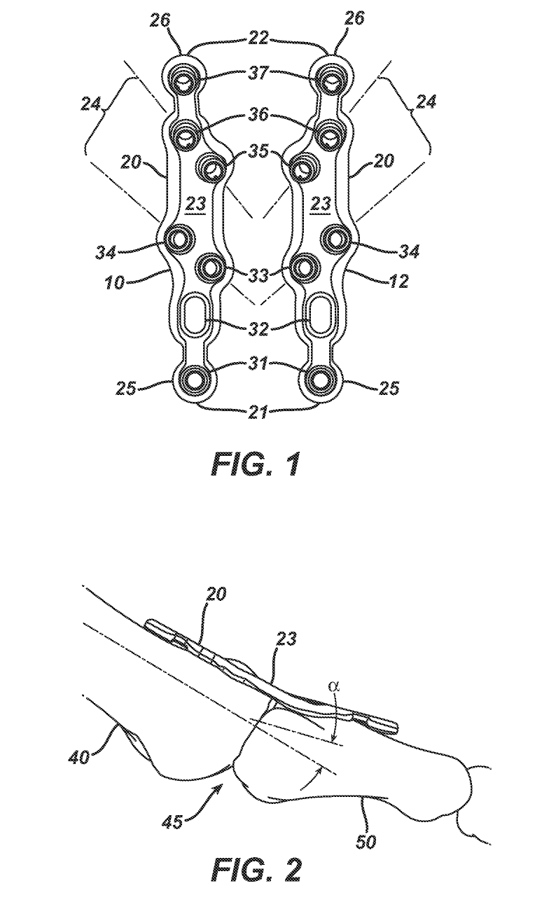

FIG. 1 depicts a left fusion plate (10) and a right fusion plate (12). In this example the left and right plates (10, 12) are mirror images of one another. Each plate has a body (20) having a proximal end (21), a distal end (22), and a longitudinal axis extending between the proximal and distal ends. The body (20) may be constructed from a metal, such as a titanium or steel alloy, or other materials suitable for bone plates. A rigid joint bridge (23) is positioned intermediate the proximal end (21) and distal end (22). The bridge (23) separates the body (20) between a proximal bone portion and a distal bone portion. The proximal bone portion comprises a proximal tab (25), and the distal bone portion comprises a distal tab (26). The tabs (25, 26) are bendable to facilitate custom fitting of the plate to a patient's unique anatomy.

A plurality of screw receiving holes (31-37) extend through of the plate body (20). Holes (31-34) extend through the proximal bone portion of the plate, and holes (35-37) extend through the distal bone portion of the plate. In the present embodiment, the proximal bone portion comprises three threaded screw holes (31, 33, 34) and one slotted screw hole (32), and the distal bone portion comprises three threaded screw holes (35-37). The slotted hole (32) may be used for initial fixation of the plate to bone, but the slot (32) still allows for movement of the plate if necessary. The threaded screw holes (31, 32-37) provide locked fixed angle screw fixation to the bone. Naturally, the number, type and configuration of the screw holes may vary from those disclosed in this embodiment. As shown in this embodiment, the threaded holes (31, 33-37) may optionally be preloaded with removable drill guides.

The distal most pair of the holes (33, 34) of the proximal bone portion defines a first transverse axis at an oblique angle relative the longitudinal axis. The proximal most pair of the holes (35, 36) of the distal bone portion defines a second transverse axis at an oblique angle relative the longitudinal axis. The first and second transverse axes may be substantially parallel to one another (i.e., within about 10 degrees). The transverse axes are preferably between about 30 and about 70 degrees from the longitudinal axis, more preferably between about 40 and about 65 degrees from the longitudinal axis, and most preferably between about 50 and about 60 degrees from the longitudinal axis. The first and second transverse axes define a channel (24) to receive a screw traversing the joint. Joint traversing screws are optional based on the patient need and surgeon discretion, and if used may be inserted proximally in the channel (24) and preferably will remain bounded within the first and second transverse axes.

FIG. 2 depicts a lateral view of a left plate (10) positioned on the left metatarsal (40) and phalanx (50) bones. The bridge (23) spans the metatarsal-phalangeal joint (45). The proximal and distal bone portions of the plate body (20) are angled relative one another to facilitate a desired fusion angle. As shown in this example, the proximal and distal bone portions may have a nonlinear dorsal angle. Preferably the metatarsal (40) and phalanx (50) fusion angle (a) will be between about 8 and about 20 degrees of dorsiflexion, more preferably between about 11 and about 17 degrees, and most preferably about 14 degrees. Preferably the metatarsal (40) and phalanx (50) fusion angle will be between about 1 and about 9 degrees of valgus, more preferably between about 3 and about 7 degrees, and most preferably about 5 degrees.

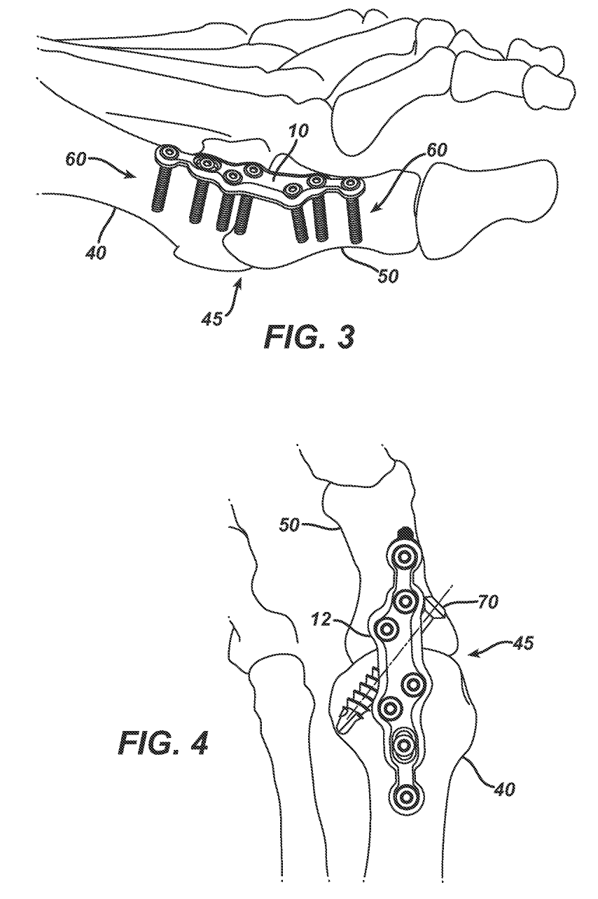

FIG. 3 depicts a perspective view of a left plate (10) positioned on the left metatarsal (40) and phalanx (50) bones with a plurality of screws (60) extending through the holes and securing the plate (10) to the bones. In this embodiment the joint fusion construct does not include a screw traversing the joint (45).

FIG. 4 depicts an alternative fusion construct with a right plate (12) positioned on the left metatarsal (40) and phalanx (50) bones with a plurality of screws extending through the holes and securing the plate (10) to the bones. The orientation of a joint traversing screw (70) may vary based on surgeon discretion. This embodiment includes a joint traversing screw (70) extending under the plate (12), starting from the phalanx (50), extending medially and proximally through the joint (45), and into the metatarsal (40). Some alternative orientations include medial-distal, lateral-proximal, and lateral-distal directions. Left and right plates (10, 12) may be interchanged depending upon the surgeon's preference for the orientation of joint traversing screw (70). The joint traversing screw (70) may be inserted before, after, and between placements of the plate screws (60).

Having shown and described various embodiments and examples of the present invention, further adaptations of the methods and devices described herein can be accomplished by appropriate modifications by one of ordinary skill in the art without departing from the scope of the present invention. Several of such potential modifications have been mentioned, and others will be apparent to those skilled in the art. For instance, the specific materials, dimensions, and the scale of drawings will be understood to be non-limiting examples. One with ordinary skill in the art will also appreciate that MTP joint fusion is only one example of a joint fusion surgery, and the teachings of the present disclosure can also be applied to other joints.

Accordingly, the scope of the present invention should be considered in terms of the following claims and is understood not to be limited to the details of structure, materials, or acts shown and described in the specification and drawings.

1. A construct for fusing a joint between a proximal bone and a distal bone, the construct comprising:
a plate body having a proximal end, a distal end, and a longitudinal axis extending between the proximal and distal ends;
a rigid joint bridge on the plate body intermediate the proximal and distal ends, the bridge separating the plate body between a proximal bone portion and a distal bone portion;
a plurality of screw receiving holes extending through the proximal bone portion of the plate, the distal most pair of the holes defining a first transverse axis at an oblique angle relative the longitudinal axis;
a plurality of screw receiving holes extending through the distal bone portion of the plate, the proximal most pair of the holes defining a second transverse axis at an oblique angle relative the longitudinal axis;
a plurality of screws extending through the holes and attaching the plate body to the proximal and distal bones; and
a joint screw traversing the joint between the first and second transverse axes.

2. The construct of claim 1, wherein proximal and distal bone portions are angled relative one another.

3. The construct of claim 2, wherein the distal bone is fused at about 8 and about 20 degrees of dorsiflexion.

4. The construct of claim 2, wherein the distal bone is fused at about 1 and about 9 degrees of valgus.

5. The construct of claim 1, wherein the proximal bone is the first metatarsal and the distal bone is the first phalanx.

6. The construct of claim 1, wherein the first and second transverse axes are between about 30 and about 70 degrees from the longitudinal axis.

7. A fusion plate for a joint between a proximal bone and a distal bone, the fusion plate comprising:
a plate body having a proximal end, a distal end, and a longitudinal axis extending between the proximal and distal ends;
a rigid joint bridge on the plate body intermediate the proximal and distal ends, the bridge separating the plate body between a proximal bone portion and a distal bone portion;
a plurality of screw receiving holes extending through the proximal bone portion of the plate, the distal most pair of the holes defining a first transverse axis at an oblique angle relative the longitudinal axis; and
a plurality of screw receiving holes extending through the distal bone portion of the plate, the proximal most pair of the holes defining a second transverse axis at an oblique angle relative the longitudinal axis;
wherein the first and second transverse axes define a channel to receive a screw traversing the joint.

8. A method for fusing the metatarsal-phalangeal joint, comprising traversing the metatarsal-phalangeal joint with a joint screw;
obtaining the fusion plate of claim 7;
placing the fusion plate on the metatarsal and the phalanx such that the bridge spans the metatarsal-phalangeal joint, and the joint screw is positioned within the channel;
obtaining a plurality of screws sized for the screw receiving holes;
attaching the proximal bone portion to the metatarsal with one or more screws;
attaching the distal bone portion to the phalange with one or more screws; and
obtaining a joint screw.

9. The method of claim 8, wherein the step of traversing is performed after the steps of attaching the plate.

10. The fusion plate of claim 7, wherein the distal most and proximal most pairs of holes are threaded fixed angle screw holes.

11. The fusion plate of claim 7, wherein the proximal bone portion comprises a slotted screw receiving hole.

12. The fusion plate of claim 7, wherein the proximal bone portion comprises a bendable proximal tab comprising a screw receiving hole.

13. The fusion plate of claim 7, wherein the distal bone portion comprises a bendable distal tab comprising a screw receiving hole.

14. The fusion plate of claim 7, wherein the screw receiving holes consist of three threaded screw holes and one slotted screw hole in the proximal bone portion, and three threaded screw holes in the distal bone portion.

15. The fusion plate of claim 7, wherein the first and second transverse axes are between about 50 and about 60 degrees from the longitudinal axis.

16. The fusion plate of claim 15, wherein the first and second transverse axes are substantially parallel to one another.

17. A fusion plate for the metatarsal-phalangeal joint, the fusion plate comprising:
a plate body having a proximal end, a distal end, and a longitudinal axis extending between the proximal and distal ends;
a rigid joint bridge on the plate body intermediate the proximal and distal ends, the bridge separating the plate body between a metatarsal portion and a phalanx portion, the metatarsal and phalanx portions being angle relative one another in the dorsal direction;
three or more screw receiving holes extending through the metatarsal portion of the plate, the distal most pair of the holes defining a first transverse axis at an oblique angle between about 50 and about 60 degrees relative the longitudinal axis; and
three of more of screw receiving holes extending through the phalanx portion of the plate, the proximal most pair of the holes defining a second transverse axis substantially parallel with the first transverse axis;
wherein the first and second transverse axes define a channel adapted to receive a screw traversing the joint.

18. The fusion plate of claim 17, wherein the metatarsal portion of the plate consists of three threaded fixed angle screw receiving holes and a single slotted screw receiving hole.

19. The fusion plate of claim 18, wherein the phalanx portion consists of three threaded fixed angle screw receiving holes.
